(19) United States
(12) Patent Application Publication
(10) Pub. No.: US 2019/0314567 A1
(43) Pub. Date: Oct. 17, 2019

(54) Intravascular Membrane Oxygenator Catheter Systems and Methods

(71) Applicant: Duke University, Durham, NC (US)

(72) Inventors: Tobias Straube, Durham, NC (US); Ken Gall, Durham, NC (US); Bruce Klitzman, Durham, NC (US); Ira Cheifetz, Durham, NC (US); Roy Glenn Morris, Conway, AR (US); Travis Vesel, Durham, NC (US); Jennifer Chien, Durham, NC (US)

(21) Appl. No.: 15/950,517

(22) Filed: Apr. 11, 2018

(51) Int. Cl. A61M 1/16

(52) U.S. Cl. CPC A61M 1/1698; A61M 2202/0208; A61M 2205/3368; A61M 1/363; A61M 2205/3344; A61M 2210/125; A61M 2205/3334

(57) ABSTRACT

Intravascular membrane oxygenator catheter systems and methods are disclosed, along with methods of making and using the same. A catheter system includes a catheter shaft having a wall that extends from a proximal end to a distal end along a longitudinal axis to define a lumen, a plurality of hollow fiber membrane loops each having a proximal portion positioned within the lumen of the catheter shaft and a distal portion extending beyond the lumen of the catheter shaft, the hollow fiber membrane loops are nonporous with solid walls; a pneumatic source in pneumatic communication with the hollow fiber membrane loops. The pneumatic source provides a gas containing oxygen at a pressure sufficient to cause a diffusive flux of the gas containing oxygen from an interior of the hollow fiber membrane loops to a fluid exterior to the hollow fiber membrane loops in a region of interest of a subject.

### CROSS-REFERENCE TO RELATED APPLICATIONS

N/A

### STATEMENT REGARDING FEDERALLY SPONSORED RESEARCH

N/A

### BACKGROUND

Respiratory failure can be caused by primary lung disease, such as pneumonia, or be secondary to virtually any systemic illness. This wide array of pathology makes respiratory distress, which can lead to respiratory failure, a common cause for hospitalization in neonates, children, and adults. In 2009, acute respiratory failure accounted for 1,917,910 hospitalizations in adults alone with a total predicted inpatient cost of $54 billion.

When the respiratory system begins to fail, options quickly become limited even with the capabilities of modern medicine. A mainstay of treatment for patients with severe disease remains mechanical ventilation, which involves placing a tube in patient's airway and pushing gas into the lungs. In 2005, there were 790,257 hospitalizations involving mechanical ventilation in the United States (children and adults) with the number continuing to rise. Morbidity in the form of ventilator induced lung injury, ventilator associated pneumonia, airway injury, and need for pharmacologic sedation can harm patients and prolong hospital stays. As of 2005, the total cost associated with mechanical ventilation in the United States alone was estimated at $27 billion.

When patients remain hypoxic despite maximizing mechanical ventilation, clinicians may turn to extracorporeal membrane oxygenation (ECMO). ECMO acts as an artificial lung outside the body surgically connected via inflow and outflow lines. In 2012, ECMO was deployed in over 1600 neonatal, pediatric, or adult cases for respiratory failure. Since 2013, 17,930 neonates, children, and adults with respiratory failure have been supported with ECMO per the international ELSO registry. This number likely underrepresents the actual number of patients who required ECMO since that time. Of the nearly 18,000 ECMO cases, 62% were adult patients. While ECMO can be life-saving, it is associated with significant morbidity or predicted mortality and is far more expensive than conventional mechanical ventilation. Common complications include hemorrhage due to the need for aggressive systemic anticoagulation (intracranial hemorrhage in up to 11% of patients), renal failure (15.8% of adult respiratory patients), and life-threatening infections (17.1% of adult respiratory patients). In addition to its morbidity, deploying ECMO and maintaining its use is costly. It requires a surgical procedure for placement, dedicated nursing (one-on-one staffing), and perfusion specialists around the clock for maintenance. It can only be used in an ICU with providers experienced in its use. The average cost of one ECMO run in the United States ranges from $105,034 to $335,565.

A previous experimental approach to treating respiratory failure was the IVOX catheter, which was composed of a bundle of hundreds of small microporous hollow fibers furled together to fit in the vena cava with sub-atmospheric oxygen flowing through the lumens of the fibers. While this device provided measurable gas exchange its large size (˜10-14 mm diameter in furled position), made it cumbersome and led to a 24.5% clinically recognized complication rate including bleeding during insertion, thrombophlebitis, and hemodynamic instability.

Another attempt at building a device aimed at treating respiratory failure is the PENSIL catheter, which was a 150 cm multi-lumen gas supply catheter with several thousand polypropylene blind-ended hollow fibers. The PENSIL catheter delivered only about 15-20 mL of O₂ per minute (˜10% of an adult's basal need) while taking up the entire inferior vena cava, right side of the heart, and pulmonary arteries. This device never made it to human clinical trials.

Yet another example was the Hattler Catheter which was composed of a bundle of hundreds of microporous hollow fibers with a pumping balloon in the middle. It was placed in the right atrium and generated higher gas flux but this design was cumbersome, decreased cardiac return, and never progressed to human clinical trials.

These prior intravascular devices all relied on a large surface area to deliver oxygen to, and remove carbon dioxide from, the blood via convection through their microporous hollow fiber membranes. This approach created large bulky catheters (many bigger than 16 mm in diameter and as long as 150 cm) that impaired cardiac return, were difficult to deploy, and risked clot/embolism formation, among other issues.

A solution is needed to overcome the challenges encountered by previous groups attempting to build intravascular devices aimed at treating respiratory failure. Their designs created large bulky catheters that impaired cardiac return, were difficult to deploy in regions of interest, and also risked clot/embolism formation. An intravascular device that provides oxygen directly into the bloodstream and overcomes the challenges encountered by previous groups would be a minimally-invasive, safer, and less costly alternative, or supplement to, current therapies such as mechanical ventilation and Extracorporeal Membrane Oxygenation when treating neonatal, pediatric, and adult patients with acute hypoxic respiratory failure.

### BRIEF SUMMARY

In an aspect, the present disclosure provides an intravascular oxygenation catheter that will deliver oxygen directly into the bloodstream in neonatal, pediatric, and adult patients with acute hypoxic respiratory failure regardless of etiology. The catheter will be a minimally-invasive, safer, and less costly alternative or supplement to current therapies. Given the universal goal of maintaining blood oxygenation in the critically ill, such a catheter could have endless clinical applications throughout all settings of healthcare delivery (pre-hospital, Emergency Department, ICU, OR etc.).

In an aspect, the present disclosure provides a catheter system. The catheter system includes a catheter shaft having a wall that extends from a proximal end to a distal end along a longitudinal axis to define a lumen, a plurality of hollow fiber membrane loops each having a proximal portion positioned within the lumen of the catheter shaft and a distal portion extending beyond the lumen of the catheter shaft, the plurality of hollow fiber membrane loops are nonporous with solid walls; a pneumatic source in pneumatic communication with the hollow fiber membrane loops. The pneumatic source provides a gas containing oxygen at a pressure sufficient to cause a diffusive flux of the gas containing oxygen from an interior of the plurality of hollow fiber membrane loops to a fluid exterior to the plurality of hollow fiber membrane loops in a region of interest of a subject

In some aspects, the catheter system includes a pneumatic control system in pneumatic communication with the pneumatic source and the hollow fiber membrane loops. The pneumatic control system is configured to provide pressure, temperature, and flow rate control of the pneumatic source to the hollow fiber membrane loops. The pneumatic source is an oxygen source that can provide gas containing oxygen at a pressure from 1 to 2400 pounds per square inch or above 2400 pounds per square inch.

In some aspects, the catheter system is dimensioned for intravascular placement using a percutaneous approach, and the distal end of the catheter shaft is positioned in the region of interest of a subject. The region of interest can be an inferior vena cava of the subject or a right atrium of the subject's heart.

In some aspects, the catheter system further includes a manifold within the lumen, the manifold receives the hollow fiber membrane loops and support that hollow fiber membrane loops at constant distances within the lumen. The hollow fiber membrane loops comprise an amorphous fluoropolymer material and can be arranged in parallel loops having an inlet side and a return side, each side being potted in a manifold connected to the catheter shaft. The hollow fiber membrane loops can include between 1 and 500 individual hollow fiber loops that are 254 micrometers (μm) in diameter and have a wall thickness of 25.4 micrometers (μm).

In some aspects, the catheter system delivers at least 0.1 percent or more of the subject's basal oxygen needs. In some aspects, the catheter system delivers 50 to 100 percent of the subject's basal oxygen needs.

In some aspects, the hollow fiber membrane loops are subjected to vibration induced from external energies to reduce the accumulation and adherence of bubbles to the hollow fibers allowing more oxygen to be dissolved.

In another aspect, the present disclosure provides a method of intravascular oxygenation. The method can include step a) introducing a catheter system to a region of interest in a subject. In a non-limiting aspect, the catheter system can include the catheter described above. The catheter system can include a catheter shaft having a wall that extends from a proximal end to a distal end along a longitudinal axis to define a lumen. The catheter system can also have a plurality of hollow fiber membrane loops each having a proximal portion positioned within the lumen of the catheter shaft and a distal portion extending beyond the lumen of the catheter shaft. The catheter system can also have a pneumatic source in pneumatic communication with the hollow fiber membrane loops. The method can include step b) providing a gas containing oxygen at a pressure from the pneumatic source to the plurality of hollow fiber membrane loops, thereby creating a diffusive flux causing the gas containing oxygen to diffuse from an interior of the plurality of hollow fiber membrane loops to a fluid flowing past an exterior of the hollow fiber membrane loops in a region of interest of a subject.

In some aspects, the pneumatic source of step a) can be an oxygen source. The method can further comprise providing a pneumatic control system in pneumatic communication with the pneumatic source of step a) and the hollow fiber membrane loops, the pneumatic control system configured to provide pressure, temperature, and flow rate control of the pneumatic source to the hollow fiber membrane loops. The method can also include positioning the distal end of the catheter shaft in a region of interest of a subject and placing the catheter system intravascularly using a percutaneous approach.

In some aspects, the method can further comprise delivering at least 0.1 percent or more of the subject's basal oxygen needs to the subject via the catheter system

### BRIEF DESCRIPTION OF THE DRAWINGS

FIG. 1 is a schematic illustration of a catheter system according to an aspect of the present disclosure.

FIG. 2 is a perspective view of a catheter according to an aspect of the present disclosure.

FIG. 3 is a perspective view of a pair of prototype catheters according to an aspect of the present disclosure.

FIG. 4 is a plot showing a comparison of oxygen flux as a function of average intraluminal pressure as described in the Prototypes section.

FIG. 5 is a benchtop mock vasculature apparatus for in-vitro testing, as described in the In-vitro Water Testing section.

FIG. 6 is a plot comparing oxygen transmission rates to spacing, as described in the Prototypes section.

FIG. 7 is a plot showing the effect of arranging hollow fiber membrane loops in a bulb shape on oxygen transmission, according to an aspect of the present disclosure.

FIG. 8 is a top view of the inside of a pneumatic control system and a controller according to an aspect of the present disclosure.

### DETAILED DESCRIPTION

Before the present invention is described in further detail, it is to be understood that the invention is not limited to the particular embodiments described. It is also to be understood that the terminology used herein is for the purpose of describing particular embodiments only, and is not intended to be limiting. The scope of the present invention will be limited only by the claims. As used herein, the singular forms “a”, “an”, and “the” include plural embodiments unless the context clearly dictates otherwise.

It should be apparent to those skilled in the art that many additional modifications beside those already described are possible without departing from the inventive concepts. In interpreting this disclosure, all terms should be interpreted in the broadest possible manner consistent with the context. Variations of the term “comprising”, “including”, or “having” should be interpreted as referring to elements, components, or steps in a non-exclusive manner, so the referenced elements, components, or steps may be combined with other elements, components, or steps that are not expressly referenced. Embodiments referenced as “comprising”, “including”, or “having” certain elements are also contemplated as “consisting essentially of” and “consisting of” those elements, unless the context clearly dictates otherwise. It should be appreciated that aspects of the disclosure that are described with respect to a system are applicable to the methods, and vice versa, unless the context explicitly dictates otherwise.

Numeric ranges disclosed herein are inclusive of their endpoints. For example, a numeric range of between 1 and 10 includes the values 1 and 10. When a series of numeric ranges are disclosed for a given value, the present disclosure expressly contemplates ranges including all combinations of the upper and lower bounds of those ranges. For example, a numeric range of between 1 and 10 or between 2 and 9 is intended to include the numeric ranges of between 1 and 9 and between 2 and 10.

As used herein, the terms “component,” “system,” “device” and the like are intended to refer to either hardware, a combination of hardware and software, software, or software in execution. The word “exemplary” is used herein to mean serving as an example, instance, or illustration. Any aspect or design described herein as “exemplary” is not necessarily to be construed as preferred or advantageous over other aspects or designs.

Furthermore, the disclosed subject matter may be implemented as a system, method, apparatus, or article of manufacture using standard programming and/or engineering techniques and/or programming to produce hardware, firmware, software, or any combination thereof to control an electronic based device to implement aspects detailed herein.

Unless specified or limited otherwise, the terms “connected,” and “coupled” and variations thereof are used broadly and encompass both direct and indirect mountings, connections, supports, and couplings. Further, “connected” and “coupled” are not restricted to physical or mechanical connections or couplings. As used herein, unless expressly stated otherwise, “connected” means that one element/feature is directly or indirectly connected to another element/feature, and not necessarily electrically or mechanically. Likewise, unless expressly stated otherwise, “coupled” means that one element/feature is directly or indirectly coupled to another element/feature, and not necessarily electrically or mechanically.

As used herein, the term “processor” may include one or more processors and memories and/or one or more programmable hardware elements. As used herein, the term “processor” is intended to include any of types of processors, CPUs, microcontrollers, digital signal processors, or other devices capable of executing software instructions.

As used herein, the term “memory” includes a non-volatile medium, e.g., a magnetic media or hard disk, optical storage, or flash memory; a volatile medium, such as system memory, e.g., random access memory (RAM) such as DRAM, SRAM, EDO RAM, RAMBUS RAM, DR DRAM, etc.; or an installation medium, such as software media, e.g., a CD-ROM, or floppy disks, on which programs may be stored and/or data communications may be buffered. The term “memory” may also include other types of memory or combinations thereof.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

The term “effective amount” or “therapeutically effective amount” refers to an amount of a compound, cells, etc. sufficient to effect beneficial or desirable biological and/or clinical results.

As used herein, the term “subject” and “patient” are used interchangeably herein and refer to both human and nonhuman animals.

The term “flux” or “diffusive flux” refers to Fick's diffusion laws that the flux goes from regions of high concentration to regions of low concentration, with a magnitude that is proportional to the concentration gradient (spatial derivative). In simplistic terms, the concept that a solute will move from a region of high concentration to a region of low concentration across a concentration gradient. The flux or diffusive flux can be measured as a transmission rate from the region of high concentration to the region of low concentration, in some aspects in milliliters (mL) per minute. In a non-limiting aspect, the flux or diffusive flux can be measured as a transmission rate from the inside of a device as described herein into a volume of water or a volume of blood. Flux or diffusive flux can be quantified by measuring dissolved oxygen. In fact, dissolved oxygen by definition only includes flux that is dissolved in solution and is not a bubble. However, using a dissolved oxygen (DO) probe does not indicate if there are or aren't bubbles, it just indicates the amount of oxygen that is dissolved.

The term “nonporous” refers to a solid wall that does not allow direct communication from an interior side of the nonporous wall across or through it to an exterior side of the wall, allowing molecular transport only via diffusion rather than convection. Nonporous means there are no pores, even at the nano, pico, or atto scale, and the solid wall is continuous such that the material of the solid wall has no discontinuities. The term “porous” refers to a wall having pores that allow convection from an interior side of the porous wall through the pores to an exterior side of the wall. The pores in the porous wall have a diameter at or above approximately 0.1 microns to 1 micron, the pores in the porous wall can also have a diameter of 0.05 microns to 0.1 micron or smaller. A pore could also be defined as a discontinuity in the material comprising the wall, and the term porous can encompass terms such as “microporous” since this term refers to a porous or a material having discontinuity.

By way of overview and introduction, a catheter device that can deliver oxygen directly into the bloodstream in neonatal, pediatric, and adult patients with acute hypoxic respiratory failure regardless of etiology is provided in this disclosure. As will be apparent from this disclosure the catheter device can be minimally-invasive, safer, and a less costly alternative or supplement to current therapies.

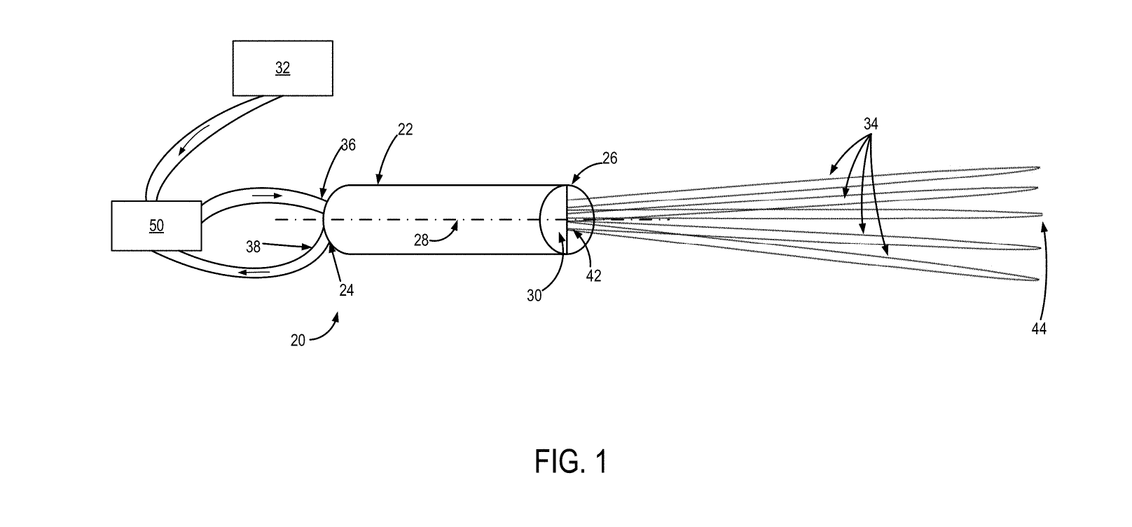

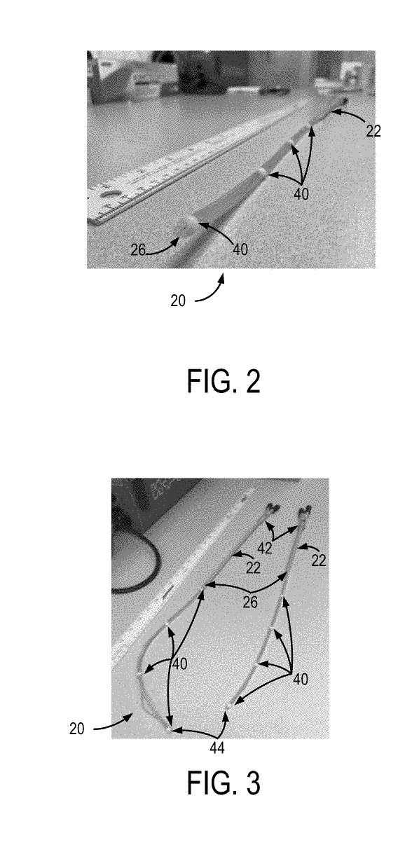

FIG. 1 shows a catheter 20 according to an aspect of the disclosure. The catheter 20 includes a catheter shaft 22 extending from a proximal end 24 to a distal end 26 along a longitudinal axis 28 to define a lumen 30. The catheter 20 is connected to a pneumatic source 32 in pneumatic communication with the catheter 20 at the proximal end 24. The pneumatic source 32 may be a high pressure source of gas containing oxygen that may be pressure, flow, and temperature regulated to supply regulated gas containing oxygen to the catheter 20. In some non-limiting aspects, the pneumatic source 32 can be a pneumatic tank, such as a medical grade oxygen tank. In other non-limiting aspects, the pneumatic source 32 can be a pneumatic pump. The pneumatic source 32 is in pneumatic communication with a pneumatic control system 50, the pneumatic control system 50 has a controller 52 and provides selective pneumatic communication to the catheter 20. The lumen 30 of the catheter shaft 22 is configured to receive a plurality of hollow fiber membrane loops (HFMs) 34 that are in pneumatic communication with the pneumatic source 32 via the pneumatic control system 50 via a pneumatic inlet 36 configured to provide high pressure gas containing oxygen to the HFMs 34. The HFMs 34 may be supported by a manifold 42 of the catheter shaft 22 that extends into the lumen 30 and provides a plurality of openings to receive the HFMs 34 such that the HFMs 34 may be potted or thermoset in the manifold 42 of the lumen 30. The HFMs 34 may also be potted or thermoset in a manifold at the proximal end 24 of the catheter shaft 22 and then travel within shaft 22 exiting at the distal end 26 through manifold 42. In some non-limiting aspects, the HFMs 34 can be potted in the manifold 42 using a high strength epoxy or other suitable materials for securely potting the HFMs 34 in the manifold 42. FIG. 2 shows a non-limiting example of the catheter 20 having a plurality of ball bearing spacers 40 which can supplement the manifold 42 attached to the catheter shaft 22, holding the HFMs 34 at constant distances apart. Other non-limiting examples of spacers include wires to space out HFMs 34, or the HFMs 34 may have intrinsic memory so that when the HFMs 34 are deployed within a vasculature of a subject, the HFMs 34 can spread out into a configuration.

The HFMs 34 can be looped such that an inlet side is connected to the pneumatic source 32 via the pneumatic inlet 36 and the inlet side can extend to the distal end 24 of the catheter 20 where the inlet side transitions to the return side of the HFMs 34. The return side of the HFMs 34 can pneumatically communicate with a pneumatic exhaust 38 that can communicate pneumatic exhaust out of the proximal end 24 of the catheter 20. The HFMs 34 can be arranged in parallel loops with both ends potted in the manifold 42. Alternatively, the HFMs 34 can have a number of non-limiting arrangements including a bulb shape, a twist, a helix, a braid pattern, and others. FIG. 1 shows a schematic illustration of the HFMs 34 spread out past the distal end 26 of the catheter shaft 22 and extending to a distal end 44 of the HFMs 34. The pressure under which the gas containing oxygen will flow is well under the bursting pressure of the HFMs 34 to ensure safety, and the gas flowing through the HFMs 34 is temperature and flow controlled. The HFMs 34 are configured to optimize non-laminar blood flow exposure to the HFMs 34 in terms of length, inner and outer diameter of HFMs 34, number of HFMs 34, outer diameter of the HFMs 34 bundle, and positioning of HFMs 34.

The pneumatic control system 50 is in pneumatic communication with the pneumatic source 32 and the pneumatic inlet 36 of the catheter 20. The pneumatic control system 50 can provide pressurized gas containing oxygen to the HFMs 34 via the pneumatic inlet 36. The pneumatic control system 50 can allow for control of the pressure and flow rate of gas containing oxygen provided to the catheter 20 via electrical communication with the controller 52. Accordingly, the pneumatic control system 50 can have a plurality of gas flow meters and pressure gauges that can be calibrated in order to ensure accurate delivery of flow and pressure to the catheter 20. The pneumatic control system 50 can also be in pneumatic communication with the pneumatic exhaust 38 at the proximal end of the catheter 20 to receive the pneumatic exhaust. Similarly, the pneumatic control system 20 can have a plurality of gas flow meters and pressure gauges that can be calibrated in order to ensure accurate measurement of the exhaust pressure and flow. The pneumatic control system 50 can regulate gas containing oxygen flow through catheter 20 allowing precise titration with continuous monitoring to match a patient's needs. In a non-limiting example, a clinician can titrate oxygen pressure and flow through the HFMs 34 in the catheter 20 to change oxygen transmission rate as needed by the patient.

The pneumatic control system 50 includes safety shut-off valves which detect a drop in pressure and will instantly stop gas flow through the HFMs 34 if a leak were to develop thereby preventing venous gas emboli formation. In one aspect, the pneumatic control system can have three (3) channels that provide high pressure oxygen to the HFMs 34. The HFMs 34 can be separately grouped or “banked” into three (3) groups that can be individually monitored for pressure through each group via the three (3) channels. If there is a sudden drop in pressure, as there would be in a catastrophic failure of one or more of the HFMs 34, the pneumatic control system 50 will sense the failure and instantly shut off gas flow through that channel (and therefore bank of HFMs 34) to prevent gas emboli (blowing gas directly into blood stream from failed hollow fiber). It is to be appreciated that the number of groups and channels described above are exemplary and any appropriate number of groups and channels can be utilized. The pneumatic control system 50 can be capable of controlling the pressure and flux of oxygen based on the inputs supplied by a physician.

The HFMs 34 receive high pressure gas containing oxygen from the pneumatic control system 50 and the gas containing oxygen flows through the HFMs 34 to provide diffusive flux of oxygen through the walls of the HFMs 34. The HFMs 34 may have small radii and accordingly can withstand high internal pressures according to LaPlace's Law since:

Wall Tension=(P)(R)

Where P is pressure and R is the radius. The ability to safely withstand high pressure means that significant flux can be achieved with only a modest surface area.

The HFMs 34 are solid wall nonporous membranes configured to deliver oxygen using exclusively diffusion rather than convection through a porous surface. Diffusion through a porous surface risks formation of high volume and large diameter bubbles which can be dangerous especially at hyperbaric pressures. The high pressure gas containing oxygen, which in some aspects may be hyperbaric, flowing through the HFMs 34 creates a driving gradient, based on Fick's laws of diffusion, diffusing oxygen out of an interior chamber of the HFMs 34 as it dissolves through the nonporous membrane of the HFMs 34 to an exterior side of the nonporous membrane and into, for example, blood flowing past the HFMs 34 in the region of interest of the subject. Generally, with a bundle of the plurality of HFMs 34, fluid flowing past can flow between the individual HFMs 34 to expose an increased surface area of HFMs 34 to fluid flowing past. In some aspects, a high partial pressure of oxygen generates the pressure gradient that greatly increases oxygen transmission from the HFMs 34 to a region of interest of the subject. The pressure gradient generated by the hyperbaric oxygen concentration in the HFMs 34 allows for a reduction in size of the catheter shaft 22 since it is not relying on an extraordinarily large surface area to generate diffusivity. As discussed in detail below, the hyperbaric nature of the catheter 20 provides improved intravascular oxygenation via the pressure gradient between the HFMs 34 and a region of interest. The catheter 20 can be adjusted as needed for the subject's size and can be sized to be used in a range of subjects, including, but not limited to neonatal, pediatric, and adult patients.

The HFMs 34 can be potted at the proximal end 24 of shaft 22 and the HFMs 34 can be bunched together inside of the shaft 22, and the HFMs 34 can extend beyond the distal end 26 of the shaft 22 where the HFMs 34 may be exposed to the region of interest (e.g. blood) such that the HFMs 34 may diffuse oxygen to the region of interest. In other aspects, the HFMs 34 may be potted in the distal end 26 of the shaft 22 at which point the shaft 22 could house an inflow and outflow connection to the manifold 42.

The catheter 20 that provides intravascular oxygenation to the subject has a reduced size due to the reliance on a large oxygen concentration gradient driving diffusion rather than convection through a porous wall. The reduced size of the catheter 20 dimensioned for insertion into a region of interest, improves biocompatibility due to reduced surface area in contact with the blood, and minimizes any hemodynamic effect. In some non-limiting aspects, the catheter shaft 22 can have a diameter of two (2) millimeters (mm) or six (6) French (Fr). In still other non-limiting aspects, the catheter shaft 22 can have a diameter between one (1) mm and three (3) mm, or 3 Fr and 9 Fr. The diameters of the catheter 20 can be between approximately 1 mm to 16 mm.

The catheter 20 is scalable such that it could be a small size appropriate for use in a neonate all the way up to a size appropriate for an adult. As such, it could range in size from one loop of HFM 34, to dozens of HFMs 34, to above 100 HFMs 34. In a non-limiting aspect, the catheter 20 may have twenty two (22) hollow fiber membrane loops 34 potted in the manifold 42 of the catheter shaft 22. In another non-limiting aspect, any appropriate number of HFMs 34 can be positioned in the catheter 20, for example between ten (10) and thirty (30) HFMs 34 can be located in the catheter 20. In a non-limiting aspect, each individual HFM 34 can be 254 micrometers (μm) in diameter and have a wall thickness 25.4 μm. In a non-limiting aspect, each individual HFM 34 can be between 200 and 800 micrometers (μm), including but not limited to, between 250 and 750 micrometers (μm), between 300 and 500 micrometers (μm), or between 350 and 450 micrometers (μm) in diameter and have a wall thickness between 20 and 30 μm. In another non-limiting aspect, HFMs 34 can be 406.4 micrometers (μm) in diameter with a wall thickness of 88.9 micrometers (μm). In a non-limiting aspect, each individual HFM 35 can have a wall thickness of between 10 and 500 micrometers (μm), including but not limited to, between 25 and 400 micrometers (μm), between 50 and 250 micrometers (μm), or between 75 and 125 micrometers (μm). In other non-limiting aspect, the HFMs 34 can range from 200 micrometers (μm) to 1800 micrometers (μm) in diameter with wall thicknesses ranging from 25 micrometers (μm) to 300 micrometers (μm). Additionally, the HFMs 34 can be custom manufactured to have varying wall thickness. In some non-limiting aspects, the HFMs 34 can extend to a length of 10 cm or 30 cm in a direction parallel with the longitudinal axis 28 of the catheter 20. In some non-limiting aspects, the HFMs 34 can extend to a length between 5 cm and 20 cm or between 5 cm and 50 cm in a direction parallel with the longitudinal axis 28 of the catheter 20.

The catheter 20 may be flexible such that it can be inserted non-invasively to a region of interest in a subject. The catheter 20 is dimensioned for intravascular placement into a subject at bedside via a percutaneous approach to reach the region of interest. The catheter 20 is dimensioned for placement into the region of interest using an introducer sheath that can provide guidance for the catheter 20 to reach the region of interest. In a non-limiting example, the region of interest may be a large vessel such as the inferior vena cava, or the catheter 20 could be placed across the right atrium of the subject's heart. The catheter 20 can be placed peripherally (e.g. into the groin or neck) and maneuvered to a central venous location such as the inferior or superior vena cava or into the right atrium. The catheter 20 can also be positioned in the arterial side in select patients so that the catheter 20 could be positioned in the aorta. Additionally, the catheter 20 could be positioned in other regions of interest other than the vasculature such as intrathecal, intraperitoneal, or subcutaneously. As will be discussed in more detail below catheter 20 is manufactured from biocompatible material(s) and can be disposable. In some aspects, the catheter 20 can be placed surgically, for example, in the inferior or superior vena cava. The hyperbaric nature of the catheter 20 provides improved intravascular oxygenation via the pressure gradient between the HFMs 34 and the region(s) of interest discussed above.

As discussed above, the catheter 20 can be used for intravascular oxygenation of a region of interest in a subject by oxygenating the blood in a patient, which may be a critically ill patient with sick and failing lungs. The catheter 20 is dimensioned for insertion intravascularly and diffuses oxygen into the blood that flows past it due to the hyperbaric nature of the catheter 20, offloading much of the work of the patient's lungs and supporting the patient as the lungs heal from the underlying disease. The catheter 20 can maintain adequate oxygenation in patients with both acute and chronic lung diseases, and can be an adjunct to mechanical ventilation (or could be used by itself) and may replace or delay the need for Extracorporeal Membrane Oxygenation. The Intravascular Membrane Oxygenator Catheter overcomes these limitations through a design that is easy to deploy, simple to use, and delivers a clinically significant amount of oxygen to the patient. In some aspects, the catheter 20 can deliver any amount of oxygen that could be useful to the patient. Accordingly, the catheter 20 may deliver at least 0.1 percent of the patient's oxygen needs. The catheter 20 may deliver between 0.1 percent and one percent of the patient's oxygen needs. In some aspects, the catheter 20 may deliver greater than one (1) percent of the patient's oxygen needs. For example, the catheter 20 may deliver one (1) percent to five (5) percent of the patient's oxygen needs. In some aspects, the catheter 20 may deliver five (5) percent to twenty-five (25) percent of the patient's oxygen needs. In some aspects, the catheter 20 may deliver twenty five (25) percent or more of a patient's basal oxygen needs in a catheter 20 configured to be sized to easily fit intravascularly into a region of interest. Furthermore, the catheter may deliver 50-100% of the patient's basal oxygen demand. In some cases, the catheter may deliver greater than 100% of the patient's basal oxygen demand.

Using known oxygen permeabilities and tensile strengths of various materials (e.g. polymers), a small bundle of HFMs 34, which may be equal or smaller in size than current central intravenous catheters, placed under high pressures (possibly up to 1000 PSI, up to 2400 PSI, or greater) can diffuse a clinically significant amount of oxygen at well under the bursting pressures of the HFMs 34.

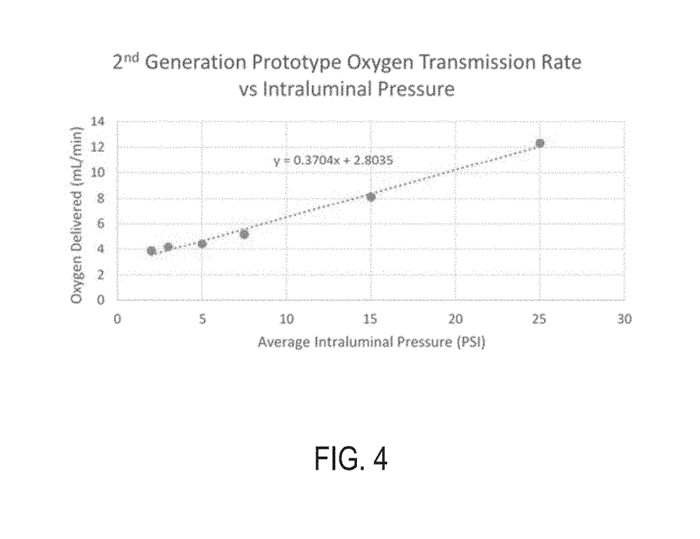

As seen in FIG. 4, experimental results show the oxygen transmission rate from the HFMs 34 in a flow through system (one in which fluid flows past the bundle of HFMs 34) increases linearly as the oxygen partial pressure within the HFMs 34 increases when in water. This data correspondingly shows the catheter 20 via the HFMs 34 may provide 25% to 50% of a patient's basal oxygen metabolic needs in a device, such as the catheter 20, that easily fits intravascularly at intraluminal pressures less than 200 PSI. Studies have shown that the oxygen gas exchange rate from a hollow fiber to blood is about 2.4 times than that in water only furthering our expected oxygen transmission rates.

In some aspects, incorporating a component of mechanical energy by vibrating the HFMs 34 in the catheter 20 at high frequencies (e.g. up to 1000 Hz) can help resolve supersaturation of blood immediately surrounding the HFMs 34 and the catheter 20. The vibration will disturb blood boundary layer formation around each individual HFM allowing more oxygen to be dissolved. Moreover, even if microbubbles form on the surface of the HFM they will be disrupted by the mechanical energy and will be swept away into the bloodstream to be dissolved prior to coalescing into larger clinically significant bubbles.

Additionally, thrombus formation would decrease the surface area of blood in contact with the HFMs 34 thereby limiting oxygen transmission and potentially also showering micro-emboli downstream. While the HFMs 34 are biocompatible, further prevention of thrombus formation could be needed. An approach to prevent thrombus formation can be coating the HFMs 34 with an antithrombotic material. One example is the well characterized process of using a cationic surfactant, tridodecyl methylammonium chloride (TDMAC), to adsorb to the surface of the polymeric catheter, then ionically bind heparin. These surface antithrombotic coatings of PTFE have been used successfully, and other modifications could be employed.

In some aspects, the catheter 20 can be placed in stagnant fluid such that the fluid around the catheter 20 is not flowing past at any constant or pulsed rate. In some non-limiting aspects, the above-described catheter 20 can be modified as a device that can have applications outside of medicine such as use in a bioreactor or other restricted reservoirs.

### Materials/Manufacture

The catheter 20 and corresponding components can be manufactured from a variety of materials. In some non-limiting aspects, the material for the HFMs 34 can be a type of polymer such as Teflon Amorphous Fluoropolymer (AF), polypropylene, polyacetylene polymethylpentene, polyethylene, polycarbonate, or polystyrene, which provide gas diffusion sufficient for use in the subject application.

The HFMs 34 may be manufactured from amorphous polytetrafluoroethylene (PTFE). In one non-limiting aspect, the HFMs 34 may be manufactured from the amorphous polymer Teflon® Amorphous Fluoropolymer (AF) 2400 due to its biocompatibility, high oxygen transmission rate, nonporous nature, and excellent mechanical strength (bursting pressure over 2400 PSI for our HFM in their current dimensions). Teflon AF can be manufactured in the form of the HFMs 34 in a scalable manner in accordance with current good manufacturing procedures (CGMP) and FDA requirements. Prototype Teflon AF HFMs 34 using tubing that is 406.4 μm in diameter with an 88.9 μm wall thickness have also been used.

### Methods

In an aspect, a method of intravascular oxygenation is also provided. The method can include step a) providing a catheter system to a region of interest in a subject. In a non-limiting aspect, the catheter system can include the catheter 20 described above. The catheter system can include a catheter shaft having a tubular wall that extends from a proximal end to a distal end along a longitudinal axis to define a lumen. The catheter system can also have a plurality of hollow fiber membrane loops positioned within the lumen of the catheter shaft; the hollow fiber membrane loops and the tubular wall each having a nonporous membrane. The catheter system can also have a pneumatic source in pneumatic communication with the hollow fiber membrane loops. The method can include step b) providing high pressure oxygen from the pneumatic source to the hollow fiber membrane loops. The method can include step c) creating a diffusive flux causing oxygen to diffuse from the catheter system to a region of interest of a subject.

In some aspects, the pneumatic source of step a) can be an oxygen source. The method can further comprise providing a pneumatic control system in pneumatic communication with the pneumatic source of step a) and the hollow fiber membrane loops, the pneumatic control system configured to provide pressure, temperature, and flow rate control of the pneumatic source to the hollow fiber membrane loops. The method can also include positioning the distal end of the catheter shaft in a region of interest of a subject and placing the catheter system intravascularly using a percutaneous approach.

In some aspects, the method can include delivering one (1) to twenty five (25) percent of the subject's basal oxygen needs. In some aspects, the method can include delivering twenty five (25) percent or more of the subject's basal oxygen needs to the subject via the catheter system. In some aspects, the method can include delivering 50 to 100 percent of the subject's basal oxygen needs.

### Prototypes

A technique to pot or thermoset the HFMs in a manifold which can connect to a high-pressure oxygen gas source was developed. First generation prototypes were built using this technique by potting HFMs to an inflow and outflow gas source in a 12 Fr configuration. These prototypes were tested in-vivo in a porcine model successfully deploying them into the inferior vena cava using a combination of introducer and retractable sheaths. The swine tolerated the catheter deployment without physiologic compromise as evidenced by stable cardiac output, blood pressures, and heart rate.

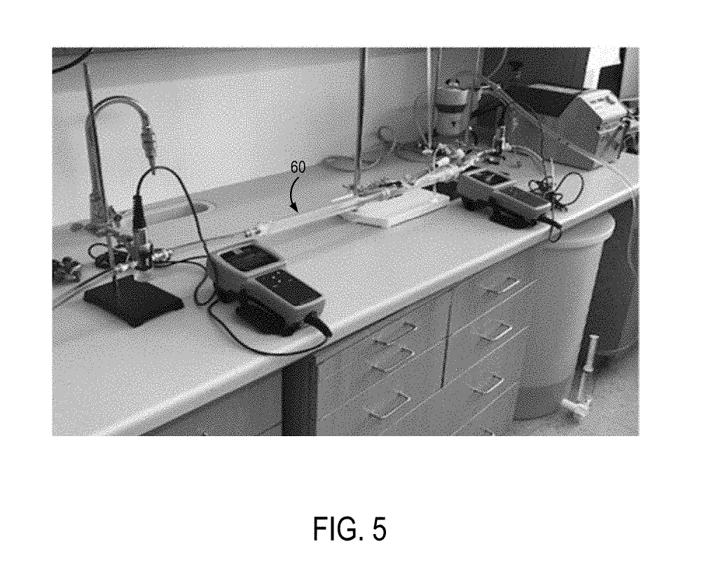

Initial proof-of-concept work was completed by testing second generation prototypes in an in-vitro benchtop mock vasculature system having an extracorporeal membrane oxygenation (ECMO) pump (e.g. Sorin Medical Group London, England) and dissolved oxygen probes (e.g. YSI 550 Yellow Springs, Ohio) in line with our custom made vinyl intravenous catheter (IVC), as shown in FIG. 5.

FIGS. 2 and 3 show a second generation prototype consisting of 1080 cm of exposed Teflon AF 2400 HFM bundled into 30 cm HFMs (which ultimately could be compressed down into a 3 mm or 9 Fr diameter configuration) delivered 12.3 mL of O₂ per minute into water at an average intraluminal pressure of 25 PSI. In blood, the prototype is expected to deliver about of 30 mL of O₂ per minute at this pressure, which is approximately 17% of a teenager's basal metabolic need. This prototype also demonstrated that oxygen transmission rates increase linearly as intraluminal oxygen partial pressure increases as shown in FIG. 4.

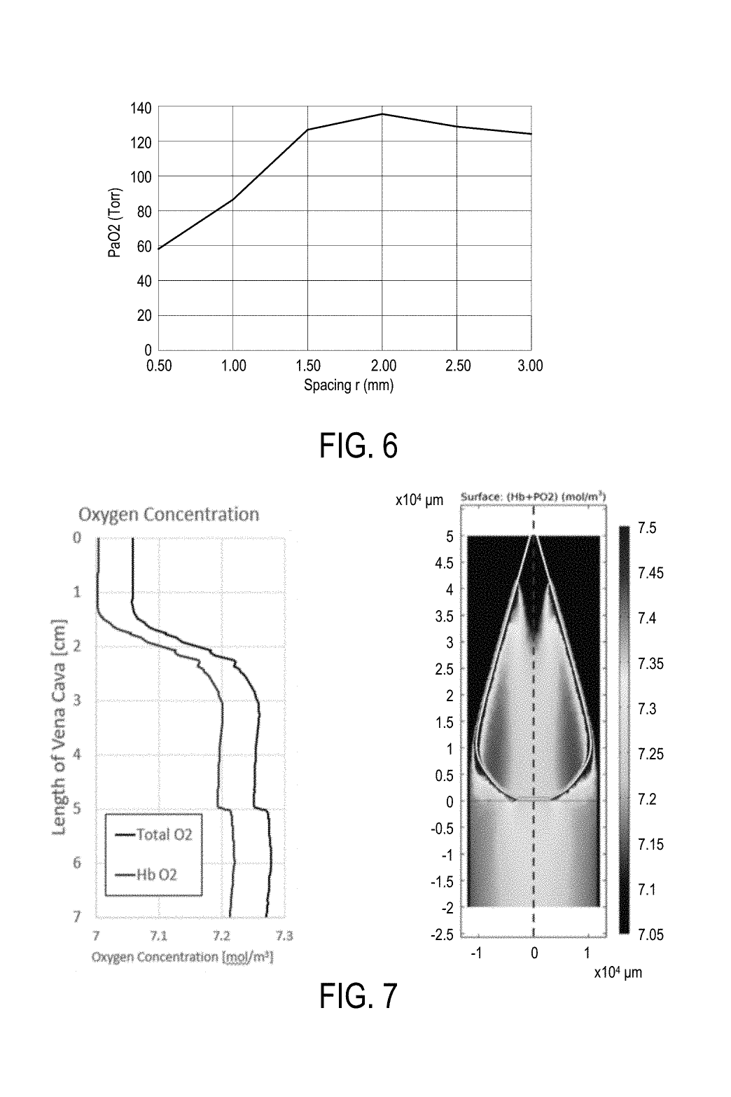

Additionally, a computer simulation was used to help identify the optimal configuration of HFMs to maximize oxygen transmission. Using our oxygen transmission rates in water, the computer simulation identified that there is no benefit to spreading out the HFMs farther apart than 2 mm within the catheter bundle configuration as seen in FIG. 6. Additionally, computer simulation showed that spreading the fibers out in a bulb shape was more efficient than having an equal surface area of HFMs in parallel as seen in FIG. 7.

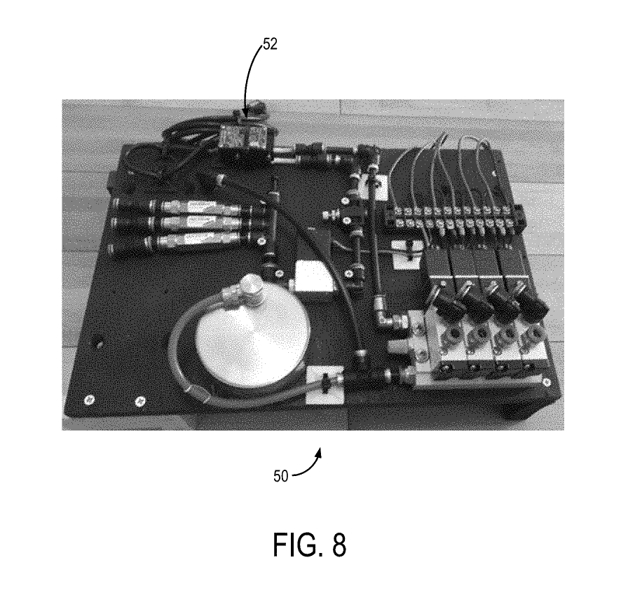

An exemplary pneumatic control system 50 is shown in FIG. 8, which can connect to the disposable intravascular oxygenation catheter(s) 20. The pneumatic control system 50 is designed to control oxygen pressure and flows through the HFMs 34 in the catheter 20 allowing clinicians to precisely titrate the oxygenation to the patients' needs. Importantly, the pneumatic control system 50 includes safety shut-off valves which detect a drop in pressure and will instantly stop gas flow through the HFMs if a leak were to develop thereby preventing venous gas emboli formation.

A number of tests have been developed in order to determine the diffusivity/oxygenation rate and overall effectiveness of the catheter 20 with the HFMs 34 as described above. These tests are not meant to be limiting, rather to be exemplary test methods.

### In-Vitro Water Testing

Four more 2^{nd} generation prototypes will be built and tested in our in vitro bench top apparatus using water. We will build catheters using our PTFE HFM as well as PTFE spherical spacers to hold the individual fibers apart at constant distances within a 5.33 mm diameter configuration. Catheters with 4, 8, 12, and 16 thirty cm HFM loops will be built in radially evenly spaced cross-sectional configurations. These catheters, in conjunction with our already completed 2^{nd} generation prototype catheter (18 loops) will be used to determine if increasing the amount of exposed HFM surface area within a constant volume (fiber density) increases the steady state O₂ delivery with a linear or negative exponential relationship according to the formula: Flux=C(1−e^{-kn}), where C is the maximal flux, k is a transfer constant, and n is the number of fibers. The 4 loop prototype will be placed in our apparatus using the 10 mm mock IVC. Water will flow through the IVC and past our catheter at 4 L/min using our Sorin ECMO pump (Sorin Group, Munchen, Germany). We will measure pre- and post-catheter dissolved O₂ using our two probes in series (YSI 550 Yellow Springs, Ohio). We will measure dissolved O₂ at intraluminal pressures of 0-25 PSI at 2 PSI increments using a manual pressure regulator to deliver O₂ from a tank. Since this apparatus measures dissolved O₂ after one pass of water as it flows past the catheter (not a circuit), there is no need to wait for the system to reach a steady state. All runs will be monitored for bubble formation using a Sorin Sensor Bubble Detector to objectively assess bubble formation. O₂ transmission rate will be reported in mL of O₂ per min. Bubble detection reporting will be binary with either bubbles or no bubbles detected. Fiber density will be reported as number of fibers per cross-section mm². The experiment will be repeated three times using the 4 loop prototype averaging O₂ transmission rates at each incremental PSI. Once completed, the process will repeat for the 8, 12, 16, and 18 loop prototypes.

The in-vitro water testing will answer several important questions further characterizing the Teflon HFM. These experiments will determine if fiber density parameters will need to be taken into account in order to maximize the oxygen transmission efficiency of our catheters. While this relationship will initially be defined in water, it would confirm the need for further testing in blood if the relationship is negatively exponential.

Learning the relationship between HFM density and oxygen transmission will help determine the optimal amount of HFM loops needed for a catheter with specific oxygen delivery goals. If adding more loops has diminishing returns, a smaller catheter may be able to deliver similar clinically significant amount of oxygen.

Sweeping from 1-25 PSI with each prototype will demonstrate if higher flux from the increased oxygen driving pressure is of the same benefit with high fiber density as it is with low fiber density due to neighboring fiber interactions. This will answer if maximum operating pressures in blood must be found in the future which differ dependent on fiber densities of subsequent catheters.

### In-Vitro Blood Testing

We will experimentally determine the O₂ transmission rate of our 2^{nd} generation prototype catheter in bovine blood. A modified Sorin S5 cardiopulmonary bypass circuit (Sorin Group, Munchen, Germany) with a Maquet ECMO oxygenator (Maquet, Rastatt, Germany) and a 10 mm custom-made mock IVC in series will be used. We will prime this circuit with bovine blood (˜1.5 L) and place the ECMO ‘oxygenator’ distal to our catheter to deoxygenate the returning blood in the circuit using nitrogen sweep gas. We will measure oxygenation via pre- and post-catheter blood partial pressure of O₂ (pO₂) and hemoglobin saturation measurements using a blood gas analyzer. We will test this prototype at a constant blood flow (4 L/min) at various intraluminal pressures (0-200 PSI stepwise at 2 and then 10 PSI intervals) using a manual pressure regulator to deliver O₂ from a tank. The blood flowing past our catheter will be kept at the same baseline pO₂ by titrating the nitrogen sweep gas in the ECMO ‘oxygenator’ as needed to deoxygenate the returning blood in the circuit. Upon turning on our catheter, or increasing to the next intraluminal O₂ pressure level, we will wait one minute as we adjust the nitrogen sweep gas in the ECMO ‘oxygenator’ allowing the circuit system to reach a steady-state. We will repeat this experiment four times recording the mean O₂ transmission rates at each respective intraluminal pressure. All runs will be monitored using a Sorin Sensor Bubble Detector (Sorin Group, Munchen, Germany) to objectively assess bubble formation. O₂ delivery will be reported in mL of O₂ per min. Bubble detection reporting will be binary with either bubbles or no bubbles detected.

Experimentally determining the relationship between oxygen transmission rates in blood compared to water with identical conditions will allows us to continue to work with water in our benchtop apparatus as we further refine and optimize subsequent prototypes.

Secondly, a maximum operating pressure for prototype will be found where oxygen transmission is maximized without bubble formation.

### Pneumatic Control System Testing

The durable pneumatic control system 50 will be tested in conjunction with our prototypes. The pneumatic control system 50 is designed to deliver oxygen flow through our device at up to 200 PSI.

First, testing the control unit oxygen pressure and flow delivery capabilities will begin by attaching the inflow/delivery and outflow ports to pressure gauges and gas flow meters to ensure accurate calibration of the pneumatic control system 50. Data can be collected from the pressure gauges and flow meters automatically or manually, and the data can be collected from the pneumatic control system 50 via LabVIEW™ software (National Instruments Austin, Tex.).

Next, the safety function of the control unit will be tested using mock catheters with three 20 cm HFM loops each individually connected to one of the pneumatic control system's 50 three inflow and outflow channels. These mock catheters will be placed underwater as oxygen flows through them. A defect will be made by cutting one of the HFM loops and the volume of escaped gas will be collected using a bubble trap (which may be an inverted graduated cylinder). This experiment can be repeated at oxygen inflow pressures ranging from 1-200 PSI collecting volume of gas leaked as the control unit instantaneously shuts down that channel after HFM failure is introduced.

Next, the second generation prototype having 18 HFMs with 1080 cm exposed HFM can be tested with the pneumatic control system 50. The prototype will be placed in the in-vitro bench top apparatus 60 using water. Oxygen transmission rate will be measured from the prototype at a constant water flow of 4L/min in a mock “IVC” 10 mm in diameter using the same protocol as previous in-vitro water experiments. The pneumatic control system 50 will be used to deliver oxygen at various pressures from 1-25 PSI while measuring oxygen transmission rates, and, using the LabVIEW™ software, the pneumatic control system 50 will also monitor the control unit's performance in terms of consistent oxygen pressure and flow delivery.

The pneumatic control system 50 will be utilized in the clinical setting to precisely deliver and control oxygen flow through the catheter 20. Evaluation of the ability of the pneumatic control system 50 to deliver (and accurately monitor) consistent oxygen pressures and flows will be done by comparing the output data generated by LabVIEW™ with manually collected data from the pressure gauges and the gas flow meters.

Regarding the safety shut-off system of the pneumatic control system 50, the volume of oxygen gas leaked as measured in the bubble trap will be quantified after an HFM induced failure. We estimate leaks to be on the order of several milliliters of oxygen prior to complete gas flow cessation. This amount of gas leak is far less than lethal circulatory arrest doses, which have been estimated to be between 200-300 mL (3-5 mg/kg) in adults. The catheter 20 is designed so that HFM leaks are “never-events” given the potential harm. Never events can be defined as adverse events that are serious, largely preventable, and of concern to both the public and health care providers for the purpose of public accountability. The Teflon AF 2400 material was chosen for the HFMs for its intrinsic mechanical strength with the bursting pressure being more than ten times the upper limit of our expected operating pressures. Nonetheless, showing that if a leak were to occur that it will be instantly stopped by the pneumatic control system is an important safety verification.

### In-vivo Pig Testing

The second generation prototypes can be tested in-vivo using a porcine model. Four porcine experiments can be used as a preliminary pilot study to quantify oxygen delivery in-vivo. The tests can be conducted based on the Duke Institutional Animal Care and Use Committee (IACUC) protocol registry number A146-15-05. Two of the studies can be approximately 8 hour runs and two studies can run 24 hours.

The pigs used in the studies can be approximately thirty-five (35) kg and can be anesthetized and intubated per protocol, including receiving three days of aspirin prior to study for anticoagulation. Test instrumentation including pulse oximetry, arterial line, Swan-Ganz catheter, and carotid Doppler US can be placed to collect baseline physiologic parameters such as heart rate, blood pressures, pulse oximetry, cardiac output, mixed venous oxygen saturation, and arterial blood gas composition. Transthoracic echocardiography can be used intermittently to monitor for bubble formation. Heparin bolus of 150 units/kg can be given in addition to a heparin infusion at time of device insertion. The catheter 20 can be inserted into the femoral vein via venotomy and advanced into the inferior vena cava similarly to previous porcine studies. Another set of baseline physiologic parameters can be recorded.

In the first pig study (˜8 hour study), the pressure will begin at a setting of one (1) PSI and physiologic parameters will be recorded at ten (10) minutes. The pressure can be increased to two (2) PSI and then another set of physiologic data will be recorded after 10 mins Pressure increases can continue in one (1) PSI increments until oxygenation is seen while monitoring for bubble formation with intermittent transthoracic echocardiography. Once the maximum safe operating pressure for the catheter 20 is found (with respect to super-saturation of venous blood and subsequent clinically significant bubble formation), the pressure increment immediately prior (1 PSI below) will be reset for an hour while continuing to collect the physiologic data every ten (10) minutes. Next, the FiO₂ provided by the ventilator will be decreased by the calculated amount of oxygen the catheter 20 is delivering at that point in time into the venous system (pre-lung) to see whether arterial oxygen saturation and oxygen partial pressures remain at their baseline. The experiment can be repeated in another pig to confirm the optimal operating pressure with regards to maximum oxygenation without adverse physiologic effects.

For the other two pig studies the operating pressure will begin at the previously determined optimal level and continue the experiment for twenty-four (24) hours. The physiologic data can be collected every thirty (30) minutes to trend efficacy across the duration of the experiments.

Continuous oxygenation has been shown in the in-vitro apparatus in water, and we plan on showing oxygenation in blood in-vitro (above) and in-vivo. Specifically, markers of oxygenation will include comparing mixed venous oxygen saturation from the Swan-Ganz catheter, which lies downstream from the catheter 20 in the inferior vena cava, upon turning the catheter 20 on to baseline mixed venous saturation and venous blood gas hemoglobin saturation. Additionally, arterial oxygen saturation and blood gas compositions can be measured. Hemodynamics can be monitored throughout the tests to ensure no compromise ensues from insertion of the catheter 20 or from the catheter 20 running. Clinically significant bubble formation can be defined as bubbles seen in the right atrium via transthoracic echocardiography in conjunction with any hemodynamic compromise at the time of or after bubble formation is initially seen. Seeing clinically significant bubbles at this stage even without oxygenation will not be defined as a failure since this will be important to identify so further solutions can be engineered going forward (e.g. the addition of mechanical energy to the HFMs).

The particular aspects disclosed above are illustrative only, as the technology may be modified and practiced in different but equivalent manners apparent to those skilled in the art having the benefit of the teachings herein. Furthermore, no limitations are intended to the details of construction or design herein shown, other than as described in the claims below. It is therefore evident that the particular aspects disclosed above may be altered or modified and all such variations are considered within the scope and spirit of the technology. Accordingly, the protection sought herein is as set forth in the claims below.

1. A catheter system comprising:
a catheter shaft having a wall that extends from a proximal end to a distal end along a longitudinal axis to define a lumen;
a plurality of hollow fiber membrane loops each having a proximal portion positioned within the lumen of the catheter shaft and a distal portion extending beyond the lumen of the catheter shaft, the plurality of hollow fiber membrane loops are nonporous with solid walls; and
a pneumatic source in pneumatic communication with the plurality of hollow fiber membrane loops;
wherein the pneumatic source is configured to provide a gas containing oxygen at a pressure sufficient to cause a diffusive flux of the gas containing oxygen from an interior of the plurality of hollow fiber membrane loops to a fluid exterior to the plurality of hollow fiber membrane loops in a region of interest of a subject, wherein the pneumatic source is configured to provide the gas containing oxygen at a pressure at or above 1 pounds per square inch (PSI).

2. The catheter system of claim 1 further comprising a pneumatic control system in pneumatic communication with the pneumatic source and the plurality of hollow fiber membrane loops, the pneumatic control system having a controller configured to provide pressure, temperature, and flow rate control of the gas containing oxygen to the plurality of hollow fiber membrane loops.

3. The catheter system of claim 1, wherein the pneumatic source is an oxygen source.

4. The catheter system of claim 1, wherein the distal end of the catheter shaft is dimensioned for positioning in the region of interest of the subject.

5. The catheter system of claim 1, wherein the catheter system is dimensioned for intravascular placement using a percutaneous approach.

6. The catheter system of claim 1, wherein the region of interest is an inferior vena cava of the subject.

7. The catheter system of claim 1, wherein the region of interest is a right atrium of the subject's heart.

8. The catheter system of claim 1, wherein the pneumatic source is configured to provide the gas containing oxygen at a pressure from 1 to 2400 pounds per square inch (PSI).

9. The catheter system of claim 1, wherein the pneumatic source is configured to provide the gas containing oxygen at a pressure at or above 2400 pounds per square inch (PSI).

10. The catheter system of claim 1 further comprising a manifold within the lumen, the manifold receives the plurality of hollow fiber membrane loops and supports the plurality of hollow fiber membrane loops at constant distances within the lumen.

11. The catheter system of claim 1, wherein the plurality of hollow fiber membrane loops comprise an amorphous fluoropolymer material.

12. The catheter system of claim 1, wherein the plurality of hollow fiber membrane loops are arranged in parallel loops having an inlet side and a return side, each inlet and return side being potted in a manifold connected to the catheter shaft.

13. The catheter system of claim 1, wherein the plurality of hollow fiber membrane loops comprise between 2 and 500 individual hollow fiber loops.

14. The catheter system of claim 1, wherein the plurality of hollow fiber membrane loops are between 100 micrometers (μm) and 1800 micrometers (μm) in diameter and have a wall thickness of between 10 micrometers (μm) and 500 micrometers (μm).

15. The catheter system of claim 1, wherein the catheter system is configured to deliver at least 0.15 mL of O₂ per minute.

16. The catheter system of claim 1, wherein the catheter system is configured to deliver between 100 ml of O₂ per minute and 200 mL of O₂ per minute.

17. The catheter system of claim 1, wherein the plurality of hollow fiber membrane loops are configured to be subjected to vibration induced from external energies to reduce the accumulation and adherence of bubbles to the plurality of hollow fiber membrane loops allowing more oxygen to be dissolved.

18. A method of intravascular oxygenation, the method comprising:
a) introducing a catheter system to a region of interest in a subject, the catheter system comprising
a catheter shaft having a tubular wall that extends from a proximal end to a distal end along a longitudinal axis to define a lumen;
a plurality of hollow fiber membrane loops each having a proximal portion positioned within the lumen of the catheter shaft and a distal portion extending beyond the lumen of the catheter shaft;
a pneumatic source in pneumatic communication with the plurality of hollow fiber membrane loops;
the plurality of hollow fiber membrane loops are nonporous; and
b) providing a gas containing oxygen at a pressure from the pneumatic source to the plurality of hollow fiber membrane loops, thereby creating a diffusive flux causing the gas containing oxygen to diffuse from an interior of the plurality of hollow fiber membrane loops to a fluid flowing past an exterior of the plurality of hollow fiber membrane loops in a region of interest of a subject, wherein the pressure is at least 1 pounds per square inch (PSI).

19. The method of claim 18, wherein the pneumatic source of step a) is an oxygen source.

20. The method of claim 18, the method further comprising: delivering at least 0.15 mL of O₂ per minute to the subject via the catheter system.

21. The catheter system of claim 1, wherein the pneumatic source is configured to provide the gas containing oxygen at a pressure at or above 25 pounds per square inch (PSI).
